(19) United States
(12) Patent Application Publication
(10) Pub. No.: US 2012/0264626 A1
(43) Pub. Date: Oct. 18, 2012

(54) MicroRNA Expression Profiling and Targeting in Chronic Obstructive Pulmonary Disease (COPD) Lung Tissue and Methods of Use Thereof

(75) Inventors: Serge Patrick Nana-Sinkam, Columbus, OH (US); Philip T. Diaz, Columbus, OH (US); Michael E. Ezzie, Columbus, OH (US)

(73) Assignee: THE OHIO STATE UNIVERSITY RESEARCH FOUNDATION, Columbus, OH (US)

(21) Appl. No.: 13/319,217

(22) PCT Filed: May 7, 2010
(86) PCT No.: PCT/US10/34022

§ 371 Date: May 2, 2012

Related U.S. Application Data
(60) Provisional application No. 61/179,702, filed on May 19, 2009.
(60) Provisional application No. 61/176,794, filed on May 8, 2009.

(51) Int. Cl. C40B 30/04; G01N 33/53; C12Q 1/68

(52) U.S. Cl. 506/9; 435/6.12; 435/6.11; 435/7.92; 204/461

(57) ABSTRACT

A method for diagnosing and staging of chronic obstructive pulmonary disease (COPD) includes measuring expression of one or more miRNAs levels in a subject suspected of suffering from COPD.

### CROSS-REFERENCE TO RELATED APPLICATIONS

This application claims the benefit of U.S. Provisional Application Nos. 61/176,794 filed May 8, 2009, and 61/179,702 filed May 19, 2009, the disclosures of which are incorporated herein by reference.

### STATEMENT REGARDING FEDERALLY SPONSORED RESEARCH

This invention was made with Government support under NIH Grant Nos. RO3 HL095425 and 60017168 and the Government has rights in this invention.

### TECHNICAL FIELD AND INDUSTRIAL APPLICABILITY OF THE INVENTION

This invention is directed to microRNA expression profiling and targeting in chronic obstructive pulmonary disease lung tissue.

### BACKGROUND OF THE INVENTION

Chronic obstructive pulmonary disease (COPD) is a term used to describe a group of progressive lung diseases encompassing emphysema and chronic bronchitis. This progressive disease is characterized by increasing shortness of breath. COPD is the fourth leading cause of death in the United States and affects over 16 million people. The disease is estimated to kill more than 100,000 Americans each year, and costs related to care of patients with COPD are significant. It causes a substantial number of patients to be disabled, physically impaired, and reduces quality of life for most people who suffer with it.

Chronic obstructive pulmonary disease (COPD) is characterized by a chronic inflammatory process and irreversible airflow obstruction with a decline in the lung function FEV1 (i.e., forced expiratory volume in 1 second). The disease may be divided into two subgroups, namely chronic bronchitis and emphysema. Chronic bronchitis is characterized by mucus hypersecretion from the conducting airways, inflammation and eventual scarring of the bronchi (airway tubes). Emphysema is characterized by destructive changes and enlargement of the alveoli (air sacs) within the lungs. Many persons with COPD have a component of both of these conditions. COPD patients have difficulty breathing because they develop smaller, inflamed air passageways and have partially destroyed alveoli.

The presenting symptoms for COPD are typically breathlessness accompanied by a decline in FEV1. Chronic bronchitis can also be diagnosed by asking the patient whether they have a “productive cough,” i.e. one that yields sputum. COPD patients are traditionally treated with bronchodilators and/or steroids and evaluated by spirometry for the presence of airflow obstruction and reversibility. If airflow obstruction is present and reversibility less than 15%, particularly in a smoker, then they are often diagnosed as having COPD.

Diagnosis and subsequent treatment of COPD is difficult since COPD is a heterogeneous disease defined as airflow obstruction that is not fully reversible and different phenotypes contribute to the severity of the disease. The evolution of COPD is a dynamic process of injury and repair that involves many mechanisms. In order to better understand the molecular pathogenesis of COPD, investigators have applied high throughput evaluation of both the transcriptosome and proteome to COPD lung tissue. To date, researchers have identified several potential candidate genes and proteins involved in cell proliferation, apoptosis, inflammation, immune response and proteolysis. The primary goal of such studies has been to identify candidate genes that may predispose a subgroup of smokers to the development of COPD.

Despite some encouraging findings in many of these studies, both genomic and proteomic platforms harbor limitations including the lack of allied studies to determine cellular localization of candidate genes, lack of reproducibility between studies, focus on extremes of disease and significant heterogeneity within the cohorts.

MicroRNAs (MiRNAs, miR) are a family of small non-coding RNAs (approximately 21-25 nt long) expressed in many organisms including animals, plants, and viruses. miRNAs target genes for either degradation of mRNA or inhibition of translation. A single miRNA may target hundreds of genes thus altering biological networks. As a result, miRNAs are attractive candidates as both biomarkers and targets for therapy. Although the function of most miRNAs remains unknown, several studies suggest that they may be integral to key biological functions including gene regulation, apoptosis, hematopoietic development and the maintenance of cell differentiation.

The identification of patterns of miRNA expression in diseases of the lung may prove useful in increasing our understanding of the molecular heterogeneity and identifying biological pathways that may be relevant to disease pathogenesis. Recently, researchers identified distinct miRNA expression profiles in lung that distinguish lung cancer tissue from normals and isolated five distinct miRNAs in adenocarcinomas that associated with survival. However, until the present invention, there have been no publications describing miRNA expression profiles in COPD.

Accordingly, there is a desire for one or more biomarkers that can identify COPD in a subject, as well as methods of providing appropriate treatment based on the stage of COPD.

### SUMMARY OF THE INVENTION

The invention is based, at least in part, on the inventors' discovery that miRNA expression profiles are detectable in lung tissue of individuals with COPD and distinguish individuals with early stage disease from those with advanced stage disease.

The inventors herein have identified distinct miRNA expression profiling in lung tissue of patients with documented COPD.

In still another aspect, there is provided herein uses of the microRNAs identified in the lung tissue of individuals with COPD in order to reveal pathways of disease pathogenesis and could serve as biomarkers for disease progression. Furthermore, the unique profile of these miRNAs can be used to demonstrate distinct networks of molecular pathways that can then be used to identify new therapeutic targets.

In a first broad aspect, there is provided herein a method for diagnosing chronic obstructive pulmonary disease (COPD) in a subject, comprising:

i) determining a level of expression one or more biomarkers selected from FIG. 1: hsa-miR-455, hsa-miR-199a*, hsa-miR-324-5p, hsa-miR-324-3p, hsa-miR-133a, hsa-miR-193a, hsa-miR-015b, hsa-miR-374, hsa-miR-017-5p, hsa-miR-203, hsa-miR-374 and hsa-miR-429, in a sample, and

ii) assessing whether the one or more of the miRNAs are expressed at a level which is higher or lower than a predetermined level, where the COPD is implicated when certain miRNAs are at, or below the level, which is lower than the predetermined level.

In certain embodiments, the method is useful as a diagnostic tool for determining disease progression, or staging, of COPD.

In certain embodiments, the method is useful as a diagnostic tool for determining disease progression, or staging, of COPD, and distinguishing one or more of: stage 1 vs stage 2; stage 1 vs stage 4; and stage 2 vs stage 4, in the subject.

In certain embodiments, the sample is selected from the group consisting of lung tissue, frozen biopsy tissue, paraffin-embedded biopsy tissue, sputum, bronchoalveolar lavage (BAL), and combinations thereof.

In certain embodiments, wherein the sample is analyzed by one or more methods selected from the group consisting of micro array techniques, PCR amplification, RNA hybridization, in situ hybridization, gel electrophoresis, and combinations thereof.

In certain embodiments, the sample is analyzed for 10 or more of the biomarkers. In certain embodiments, the sample is analyzed for 5 or more of the biomarkers. In certain embodiments, the sample is analyzed for 2 or more of the biomarkers.

In certain embodiments, the method includes correlating the expression of one or more biomarkers to the presence of stage 1 COPD in a subject.

In certain embodiments, the method includes correlating the expression of one or more biomarkers to the presence of stage 2 COPD in a subject.

In certain embodiments, the method includes correlating the expression of one or more biomarkers to the presence of stage 4 COPD in a subject.

In another broad aspect, there is provided herein a method of detecting a COPD in a biological sample comprising:

i) obtaining a sample from a subject,

ii) assaying the sample to detect the presence or absence of at least one miRNA listed in FIG. 1: hsa-miR-455, hsa-miR-199a*, hsa-miR-324-5p, hsa-miR-324-3p, hsa-miR-133a, hsa-miR-193a, hsa-miR-015b, hsa-miR-374, hsa-miR-017-5p, hsa-miR-203, hsa-miR-374 and hsa-miR-429, in a sample, and

iii) correlating the presence or absence of the miR(s) with the presence or absence of the COPD in the sample.

In certain embodiments, the method includes determining the prognosis of the subject.

In another broad aspect, there is provided herein a method for diagnosing chronic obstructive pulmonary disease (COPD) in a subject, comprising measuring expression of one or more of: hsa-miR-199a* and hsa-miR-324-3p. In certain embodiments, the method is useful as a diagnostic tool for determining disease progression, or staging, of COPD. In certain embodiments, the method is useful as a diagnostic tool for determining disease progression, or staging, of COPD, and distinguishing one or more of: stage 1 vs stage 2; stage 1 vs stage 4; and stage 2 vs 4 stage.

In another broad aspect, there is provided herein a method of determining chronic obstructive pulmonary disease (COPD) in a subject comprising:

i) obtaining a sample from the subject,

ii) analyzing the sample for the expression of one or more biomarkers, and

iii) correlating the expression of the one or more biomarkers with COPD in the subject, wherein, the biomarkers are selected from the group consisting of the miRNAs: hsa-miR-199a* and hsa-miR-324-3p.

In certain embodiments, the sample is selected from the group consisting of lung tissue, frozen biopsy tissue, paraffin-embedded biopsy tissue, sputum, bronchoalveolar lavage (BAL), and combinations thereof.

In certain embodiments, the sample is analyzed by one or more methods selected from the group consisting of micro array techniques, PCR amplification, RNA hybridization, in situ hybridization, gel electrophoresis, and combinations thereof.

In another broad aspect, there is provided herein a method of diagnosing COPD in a subject, comprising:

i) quantifying or detecting the amount of one or more miRNAs selected from FIG. 1: hsa-miR-455, hsa-miR-199a*, hsa-miR-324-5p, hsa-miR-324-3p, hsa-miR-133a, hsa-miR-193a, hsa-miR-015b, hsa-miR-374, hsa-miR-017-5p, hsa-miR-203, hsa-miR-374 and hsa-miR-429, detectable in a test sample, and

ii) comparing the amount of the at least one miRNA in the test sample with the amount present in a normal control biological sample from a normal, COPD-free, subject, where an increase in the level of the at least one miRNA being found in the test sample is indicative that the disease is either progressing or has initiated.

In another broad aspect, there is provided herein a kit for diagnosing and staging chronic obstructive pulmonary disease (COPD) in a subject, the kit comprising:

i) a substrate for holding a biological sample isolated from a human subject suspected of having COPD,

ii) an agent that specifically binds at least one or more of the diabetic proteins; and,

iii) printed instructions for reacting the agent with the biological sample or a portion of the biological sample to detect the presence or amount of at least one marker in the biological sample.

In certain embodiments, the substrate can be hydrophobic, hydrophilic, charged, or polar.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIG. 1 shows the COPD Stage Comparison for selected miRNAs.

FIG. 2A is a schematic illustration of GOLD staging that is used for COPD.

FIG. 2B is a Heat Map showing differentially expressed genes cluster the patient samples in two main groups, one contains most of the GOLD stage IV samples.

FIG. 3 shows −log(p-value) results for miR-199a.

FIG. 4 shows IPA network analysis of predicted targets for miR-199a* which revealed pathways relevant to focal adhesion, cell-cell signaling, and tissue development.

FIG. 5 shows −log(p-value) results for miR-324-3p.

FIG. 6 shows IPA network analysis of predicted targets for miR-324-3p which identified several molecular networks relevant to TLR signaling, molecular transport, cellular development, growth and proliferation.

### DETAILED DESCRIPTION OF THE PREFERRED EMBODIMENT

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The inventors herein have identified distinct patterns of miRNA expression in lung tissue of a well-defined cohort of patients with different stages of COPD. Until the present invention, there has been no identification of miRNAs in lung tissue of such patients. Now, however, the inventors herein show that unsupervised cluster analysis demonstrates the presence of miRNAs that discriminate between stages of COPD.

MiRNA profiling is a newer platform that can be useful to complement existing strategies to identify biologically relevant targets in COPD. The role of miRNA profiling of lung tissue remains unknown. While not wishing to be bound by theory, the inventors herein now believe that miRNA profiling can be used to identify distinct molecular signatures in COPD and to correlate with disease pathogenesis. These COPD signatures can complement other modalities (such as, for example, microarray/proteomic platforms) and support a personalized approach to COPD diagnosis and treatment.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified. The value of the present invention can thus be seen by reference to the Examples herein.

### EXAMPLES

Below is Table 1 showing a cohort of 28 patients with documented COPD obtained from the Lung Tissue Research Consortium.

TABLE 1
Cohort of documented COPD Cases

| | | | Pack | | | | |
|---|---|---|---|---|---|---|---|
| Stage | Gender | Age | Years | SGRQ | Height | Weight | FEV1PD |
| 4, | 60% | 50.9 | 52.8 | 61 | 66.5 | 157 | 21.2 |
| n = 10 | Males | | | | | | |
| 2, | 50% | 69.6 | 44 | 38.9 | 65.9 | 165 | 69 |
| n = 10 | Males | | | | | | |
| 1, | 50% | 68.8 | 41 | 31.9 | 65.4 | 168.7 | 89 |
| n = 8 | Males | | | | | | |

Chronic obstructive pulmonary disease (COPD) is characterized by a chronic inflammatory process and irreversible airflow obstruction with a decline in the lung function FEV1 (i.e., forced expiratory volume in 1 second). The disease may be divided into two subgroups, namely chronic bronchitis and emphysema. Chronic bronchitis is characterized by mucus hypersecretion from the conducting airways, inflammation and eventual scarring of the bronchi (airway tubes). Emphysema is characterized by destructive changes and enlargement of the alveoli (air sacs) within the lungs. Many persons with COPD have a component of both of these conditions. COPD patients have difficulty breathing because they develop smaller, inflamed air passageways and have partially destroyed alveoli.

The presenting symptoms for COPD are typically breathlessness accompanied by a decline in FEV1. Chronic bronchitis can also be diagnosed by asking the patient whether they have a “productive cough,” i.e. one that yields sputum. COPD patients are traditionally treated with bronchodilators and/or steroids and evaluated by spirometry for the presence of airflow obstruction and reversibility. If airflow obstruction is present and reversibility less than 15%, particularly in a smoker, then they are often diagnosed as having COPD.

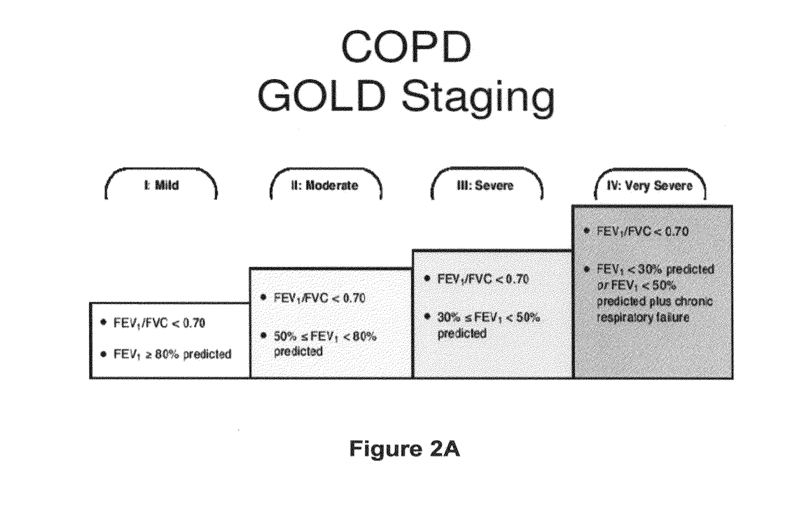

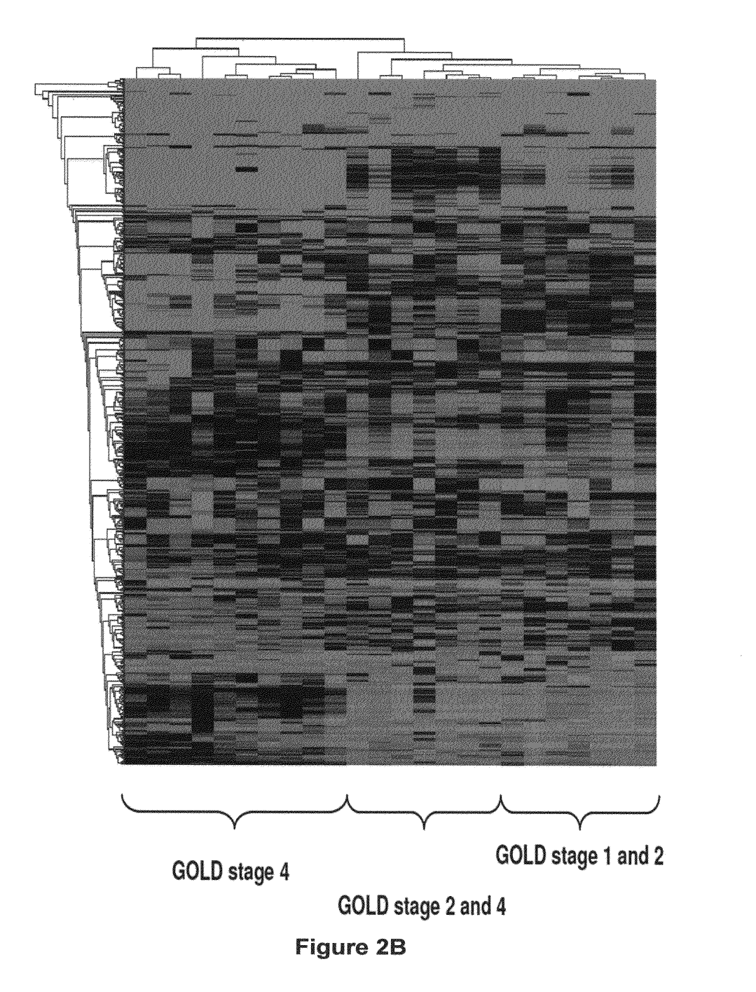

FIG. 2A a schematic illustration of the Global Initiative for Chronic Obstructive Lung Disease (GOLD) which was launched in 2001 following an NHLBI/WHO sponsored workshop which identified the need for a global response to the growing problem of COPD morbidity and mortality. Table 2 below shows the stages, the descriptions and the findings, as based on postbronchodilator FEV1).

TABLE 2
GOLD Staging System for COPD Severity

| Stage | Description | Findings (based on postbronchodilator FEV1) |
|---|---|---|
| 0 | At risk | Risk factors and chronic symptoms but normal |
| | | spirometry |
| I | Mild | FEV1/FVC ratio less than 70 percent |
| | | FEV1 at least 80 percent of predicted value |
| | | May have symptoms |
| II | Moderate | FEV1/FVC ratio less than 70 percent |
| | | FEV1 50 percent to less than 80 percent of |
| | | predicted value |
| | | May have chronic symptoms |
| III | Severe | FEV1/FVC ratio less than 70 percent |
| | | FEV1 30 percent to less than 50 percent of |
| | | predicted value |
| | | May have chronic symptoms |
| IV | Very severe | FEV1/FVC ratio less than 70 percent |
| | | FEV1 less than 30 percent of predicted value |
| | | or |
| | | FEV1 less than 50 percent of predicted value plus |
| | | severe chronic symptoms |

GOLD = Global Initiative for Chronic Obstructive Lung Disease;
COPD = chronic obstructive pulmonary disease;
FEV1 = forced expiratory volume in one second;
FVC = forced vital capacity.

FIG. 2B is a Heat Map showing differentially expressed genes cluster the patient samples in two main groups, one contains most of the GOLD stage IV samples.

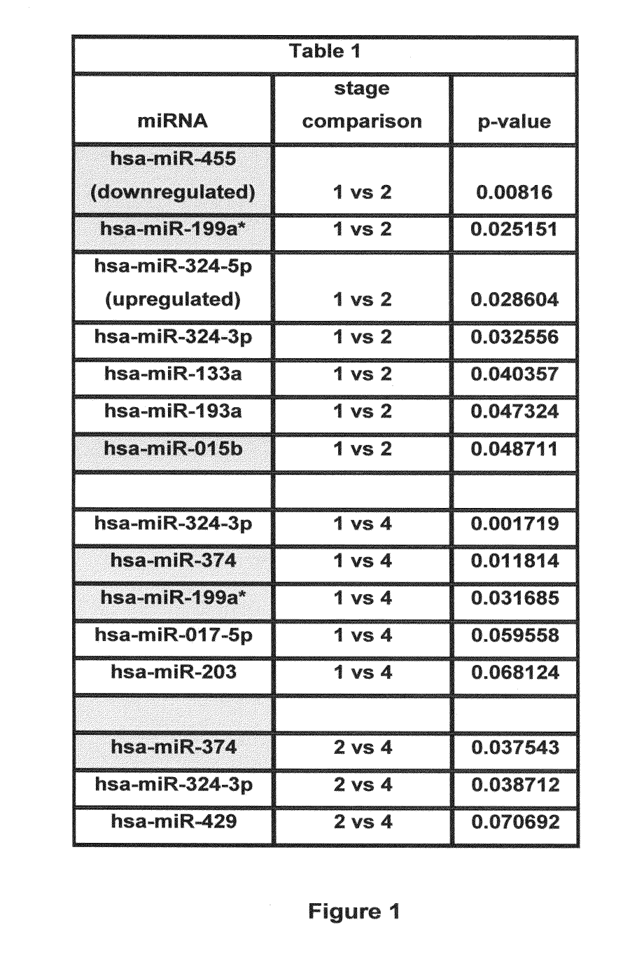

Referring now to FIG. 1, there is shown the results of high throughput qPCR used to evaluate miRNA expression. Low expression miRNAs were filtered and we used median normalization to reduce technical bias. A linear model was used for each miRNA expression dataset while adjusting for age and smoking status. Statistical tests for differential expression were then conducted between three groups with different stages of disease. P-values were obtained and the significance level was determined by controlling for the mean number of false positives.

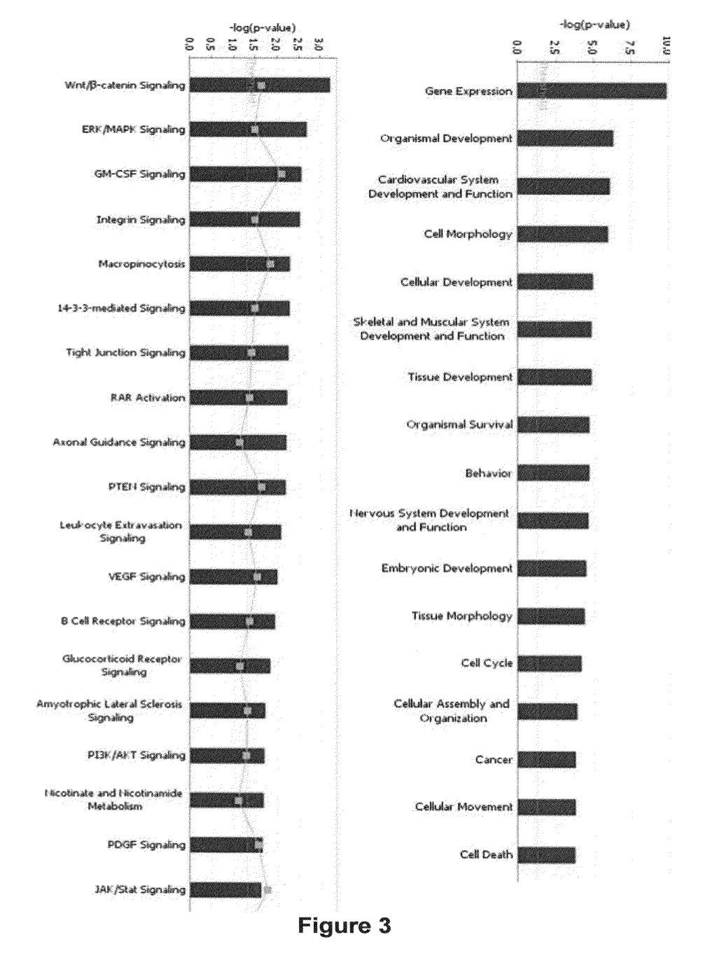

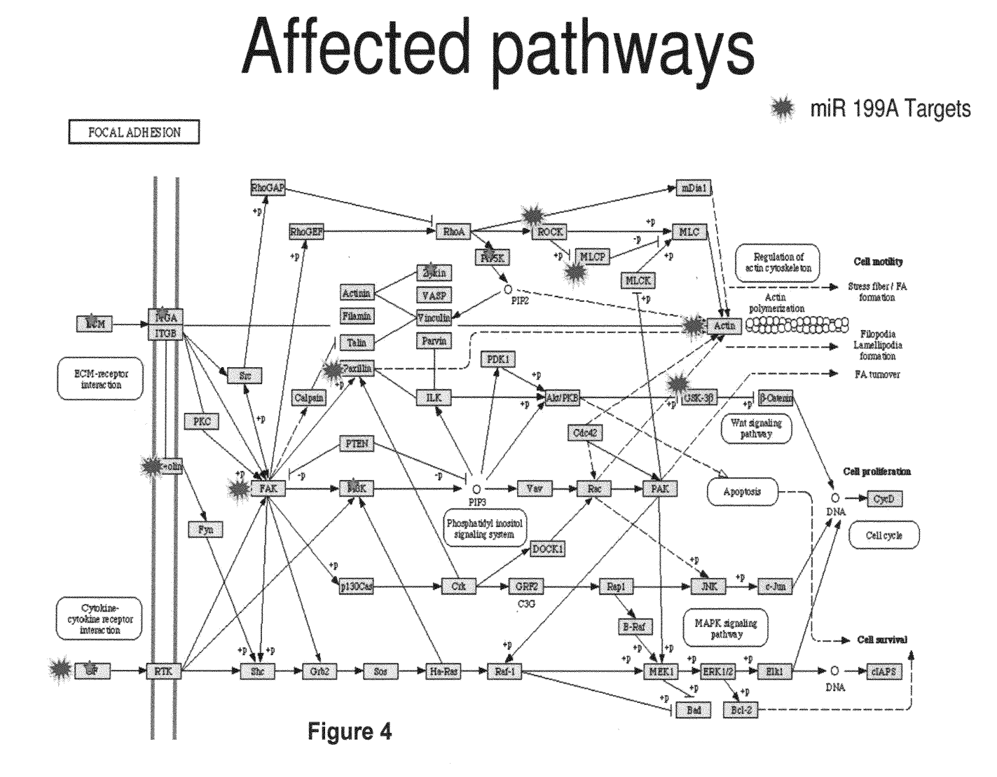

FIG. 3 shows −log(p-value) results for miR-199a. FIG. 4 shows IPA network analysis of predicted targets for miR-199a* which revealed pathways relevant to focal adhesion, cell-cell signaling, and tissue development.

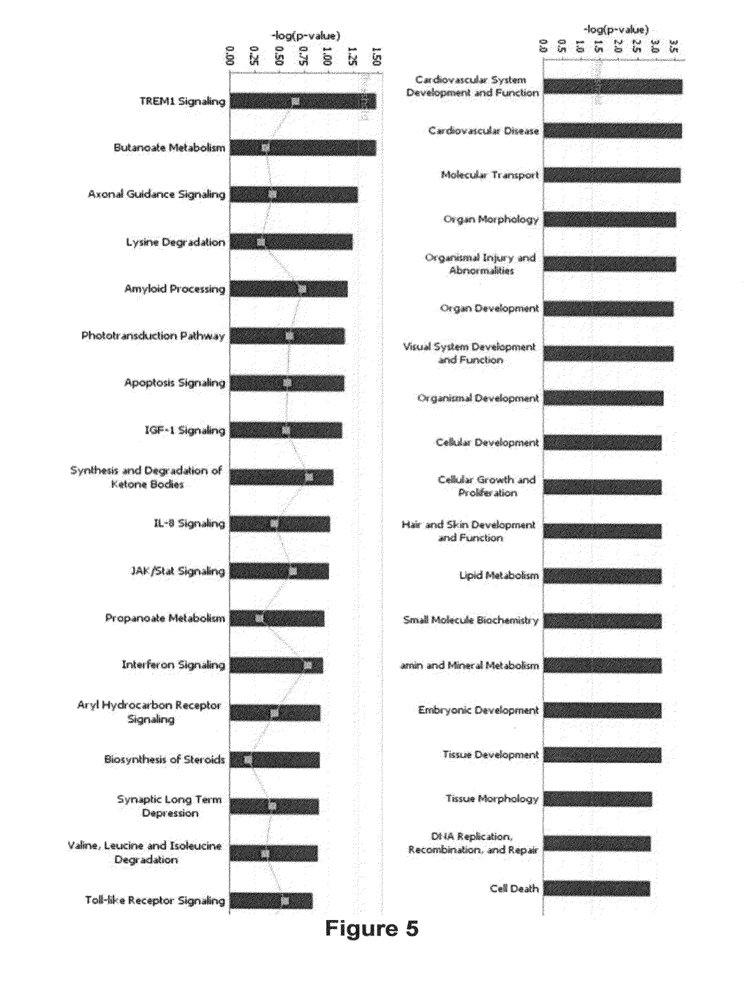

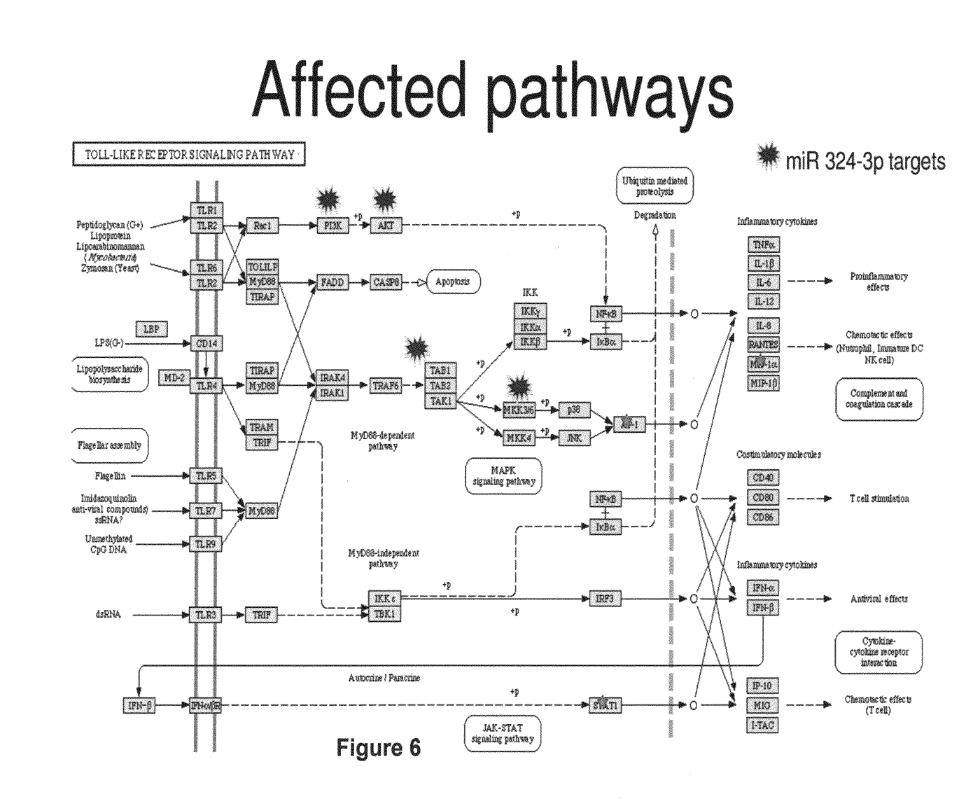

FIG. 5 shows −log(p-value) results for miR-324-3p. FIG. 6 shows IPA network analysis of predicted targets for miR-324-3p which identified several molecular networks relevant to TLR signaling, molecular transport, cellular development, growth and proliferation.

Detection Methods

Suitable techniques for determining the presence and level of expression of the biomarkers in samples are within the skill in the art. According to one such method, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters by, e.g., the so-called “Northern” blotting technique. The RNA is then immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are herein incorporated by reference. For example, the nucleic acid probe can be labeled with, e.g., a radionuclide such as ³H, ³²P, ³³P, ¹⁴C, or ³⁵S; a heavy metal; or a ligand capable of functioning as a specific binding pair member for a labeled ligand (e.g., biotin, avidin or an antibody), a fluorescent molecule, a chemiluminescent molecule, an enzyme or the like.

Probes

Probes can be labeled to high specific activity by either the nick translation method of Rigby et al, J. Mol. Biol., 113:237-251 (1977) or by the random priming method of Fienberg, Anal. Biochem., 132:6-13 (1983), the entire disclosures of which are herein incorporated by reference. The latter can be a method for synthesizing ³²P-labeled probes of high specific activity from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of biomarker levels. Using another approach, biomarker levels can be quantified by computerized imaging systems, such the Molecular Dynamics 400-B 2D Phosphorimager (Amersham Biosciences, Piscataway, N.J.).

Where radionuclide labeling of DNA or RNA probes is not practical, the random-primer method can be used to incorporate an analogue, for example, the dTTP analogue 5-(N—(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

In addition to Northern and other RNA blotting hybridization techniques, determining the levels of RNA expression can be accomplished using the technique of in situ hybridization. This technique requires fewer cells than the Northern blotting technique, and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference.

The relative number of mi-RNAs in a sample can also be determined by reverse transcription, followed by amplification of the reverse-transcribed transcripts by polymerase chain reaction (RT-PCR). The levels of RNA transcripts can be quantified in comparison with an internal standard, for example, the level of mRNA from a standard gene present in the same sample. A suitable gene for use as an internal standard includes, e.g., myosin or glyceraldehyde-3-phosphate dehydrogenase (G3PDH). The methods for quantitative RT-PCR and variations thereof are within the skill in the art.

In some instances, it may be desirable to simultaneously determine the expression level of a plurality of different biomarker genes in a sample. In certain instances, it may be desirable to determine the expression level of the transcripts of the biomarker genes correlated with COPD. Assessing specific expression levels for hundreds of biomarker genes is time consuming and requires a large amount of total RNA (at least 20 μg for each Northern blot) and autoradiographic techniques that require radioactive isotopes. To overcome these limitations, an oligolibrary in microchip format may be constructed containing a set of probe oligonucleotides specific for a set of biomarker genes. For example, the oligolibrary may contain probes corresponding to all known biomarkers from the human genome. The microchip oligolibrary may be expanded to include additional miRNAs as they are discovered.

Microchips

The microchip is prepared from gene-specific oligonucleotide probes generated from known miRNAs. For example, the array may contain two different oligonucleotide probes for each miRNA, one containing the active sequence and the other being specific for the precursor of the miRNA. The array may also contain controls such as one or more mouse sequences differing from human orthologs by only a few bases, which can serve as controls for hybridization stringency conditions. tRNAs from both species may also be printed on the microchip, providing an internal, relatively stable positive control for specific hybridization. One or more appropriate controls for non-specific hybridization may also be included on the microchip. For this purpose, sequences are selected based upon the absence of any homology with any known miRNAs.

The microchip may be fabricated by techniques known in the art. For example, probe oligonucleotides of an appropriate length, e.g., 20 nucleotides, are 5′-amine modified at position C6 and printed using suitable available microarray systems, e.g., the GENEMACHINE OmniGrid 100 Microarrayer and Amersham CODELINK activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g. 6 times SSPE/30% formamide at 25° C. for 18 hours, followed by washing in 0.75 times TNT at 37° C., for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary biomarker, in the subject sample. In an example, the labeled cDNA oligomer is a biotin-labeled cDNA, prepared from a biotin-labeled primer. The microarray is then processed by direct detection of the biotin-containing transcripts using, e.g., Streptavidin-Alexa647 conjugate, and scanned utilizing conventional scanning methods. Image intensities of each spot on the array are proportional to the abundance of the corresponding biomarker in the subject sample.

Arrays

The use of the array has one or more advantages for miRNA expression detection. First, the global expression of several hundred genes can be identified in a same sample at one time point. Second, through careful design of the oligonucleotide probes, expression of both mature and precursor molecules can be identified. Third, in comparison with Northern blot analysis, the chip requires a small amount of RNA, and provides reproducible results using as low as 2.5 μg of total RNA. The relatively limited number of miRNAs (a few hundred per species) allows the construction of a common microarray for several species, with distinct oligonucleotide probes for each. Such a tool would allow for analysis of trans-species expression for each known biomarker under various conditions.

Subjects and Samples

The subject may be a human or animal presenting with symptoms of COPD. Preferably, the subject is a human. The subject may or may not also have other lung-related disorders.

The sample obtained from the subject may be lung tissue, which can be diseased tissue or normal tissue. Alternatively, the sample may be from the subject's sputum bronchoalveolar lavage (BAL), frozen biopsy tissue, paraffin embedded biopsy tissue, and combinations thereof.

Methods for Staging

The invention further provides a method for determining the prognosis of a subject by determining whether the subject has the stage 1 vs. stage 2 vs. stage 3. vs. stage 4 COPD. The inventive method of prognosis may be utilized in lieu of current methods of prognosis. Alternatively, the inventive method may be utilized in conjunction with conventional methods of prognosis. When a combined approach is utilized, the traditional prognostic approaches may include computed tomography (CT) of the lung, magnetic resonance imaging (MRI) with contrast enhancement or angiography, and biopsy, as well as current staging systems.

The method further provides a treatment regimen that may be devised for the subject on the basis of the COPD stage in the subject. In this regard, the inventive method allows for a more personalized approach to medicine as the aggressiveness of treatment may be tailored to the stage of COPD in the subject.

In one embodiment, the invention takes advantage of the association between the biomarkers and the presence, and in certain embodiment, the stage of, COPD. Accordingly, the invention provides methods of treatment comprising administering a therapeutically effective amount of a composition comprising a reagent comprising nucleic acid complementary to at least one of the biomarkers associated with COPD. Treatment options may include traditional treatments as well as gene therapy approaches that specifically target the miRNAs described herein.

Kits

Any of the compositions described herein may be comprised in a kit. In a non-limiting example, reagents for isolating miRNA, labeling miRNA, and/or evaluating an miRNA population using an array are included in a kit. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. It may also include one or more buffers, such as reaction buffer, labeling buffer, washing buffer, or a hybridization buffer, compounds for preparing the miRNA probes, and components for isolating miRNA. Other kits may include components for making a nucleic acid array comprising oligonucleotides complementary to miRNAs, and thus, may include, for example, a solid support.

For any kit embodiment, including an array, there can be nucleic acid molecules that contain a sequence that is identical or complementary to all or part of any of the sequences herein.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being one preferred solution. Other solutions that may be included in a kit are those solutions involved in isolating and/or enriching miRNA from a mixed sample.

However, the components of the kit may be provided as dried powder(s). When reagents and/or components are provided as a dry powder, the powder can be reconstituted by the addition of a suitable solvent. It is envisioned that the solvent may also be provided in another container means. The kits may also include components that facilitate isolation of the labeled miRNA. It may also include components that preserve or maintain the miRNA or that protect against its degradation. The components may be RNAse-free or protect against RNAses.

Also, the kits can generally comprise, in suitable means, distinct containers for each individual reagent or solution. The kit can also include instructions for employing the kit components as well the use of any other reagent not included in the kit. Instructions may include variations that can be implemented. It is contemplated that such reagents are embodiments of kits of the invention. Also, the kits are not limited to the particular items identified above and may include any reagent used for the manipulation or characterization of miRNA.

It is also contemplated that any embodiment discussed in the context of an miRNA array may be employed more generally in screening or profiling methods or kits of the invention. In other words, any embodiments describing what may be included in a particular array can be practiced in the context of miRNA profiling more generally and need not involve an array per se.

It is also contemplated that any kit, array or other detection technique or tool, or any method can involve profiling for any of these miRNAs. Also, it is contemplated that any embodiment discussed in the context of an miRNA array can be implemented with or without the array format in methods of the invention; in other words, any miRNA in an miRNA array may be screened or evaluated in any method of the invention according to any techniques known to those of skill in the art. The array format is not required for the screening and diagnostic methods to be implemented.

The kits for using miRNA arrays for therapeutic, prognostic, or diagnostic applications and such uses are contemplated by the inventors herein. The kits can include an miRNA array, as well as information regarding a standard or normalized miRNA profile for the miRNAs on the array. Also, in certain embodiments, control RNA or DNA can be included in the kit. The control RNA can be miRNA that can be used as a positive control for labeling and/or array analysis.

The methods and kits of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

Array Preparation and Screening

Also provided herein are the preparation and use of miRNA arrays, which are ordered macroarrays or microarrays of nucleic acid molecules (probes) that are fully or nearly complementary or identical to a plurality of miRNA molecules or precursor miRNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters.

Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miRNA-complementing nucleic acid samples, the position of each sample can be tracked and linked to the original sample.

A variety of different array devices in which a plurality of distinct nucleic acid probes are stably associated with the surface of a solid support are known to those of skill in the art. Useful substrates for arrays include nylon, glass and silicon. The arrays may vary in a number of different ways, including average probe length, sequence or types of probes, nature of bond between the probe and the array surface, e.g. covalent or non-covalent, and the like. The labeling and screening methods described herein and the arrays are not limited in its utility with respect to any parameter except that the probes detect miRNA; consequently, methods and compositions may be used with a variety of different types of miRNA arrays

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

What is claimed is:

1. A method for diagnosing chronic obstructive pulmonary disease (COPD) in a subject, comprising the steps of:
i) determining a level of expression one or more biomarkers selected from FIG. 1: hsa-miR-455, hsa-miR-199a*, hsa-miR-324-5p, hsa-miR-324-3p, hsa-miR-133a, hsa-miR-193a, hsa-miR-015b, hsa-miR-374, hsa-miR-017-5p, hsa-miR-203, hsa-miR-374 and hsa-miR-429, in a sample, and
ii) assessing whether the one or more of the miRNAs are expressed at a level which is higher or lower than a predetermined level, where the COPD is implicated when certain miRNAs are at, or below the level, which is lower than the predetermined level.

2. The method of claim 1, useful as a diagnostic tool for determining disease progression, or staging, of COPD.

3. The method of claim 2, useful as a diagnostic tool for determining disease progression, or staging, of COPD, and distinguishing one or more of: stage 1 vs stage 2; stage 1 vs stage 4; and stage 2 vs stage 4, in the subject.

4. The method of claim 1, wherein the sample is selected from the group consisting of lung tissue, frozen biopsy tissue, paraffin-embedded biopsy tissue, sputum, bronchoalveolar lavage (BAL), and combinations thereof.

5. The method of claim 1, wherein the sample is analyzed by one or more methods selected from the group consisting of micro array techniques, PCR amplification, RNA hybridization, in situ hybridization, gel electrophoresis, and combinations thereof.

6. The method of claim 1, wherein the sample is analyzed for 10 or more of the biomarkers.

7. The method of claim 1, wherein the sample is analyzed for 5 or more of the biomarkers.

8. The method of claim 1, wherein the sample is analyzed for 2 or more of the biomarkers.

9. The method of claim 1, including correlating the expression of one or more biomarkers to the presence of stage 1 COPD in a subject.

10. The method of claim 1, including correlating the expression of one or more biomarkers to the presence of stage 2 COPD in a subject.

11. The method of claim 1, including correlating the expression of one or more biomarkers to the presence of stage 4 COPD in a subject.

12. A method of detecting a COPD in a biological sample comprising:
i) obtaining a sample from a subject,
ii) assaying the sample to detect the presence or absence of at least one miRNA listed in FIG. 1: hsa-miR-455, hsa-miR-199a*, hsa-miR-324-5p, hsa-miR-324-3p, hsa-miR-133a, hsa-miR-193a, hsa-miR-015b, hsa-miR-374, hsa-miR-017-5p, hsa-miR-203, hsa-miR-374 and hsa-miR-429, in a sample, and
iii) correlating the presence or absence of the miR(s) with the presence or absence of the COPD in the sample.

13. The method of claim 12, further comprising determining the prognosis of the subject.

14. A method for diagnosing chronic obstructive pulmonary disease (COPD) in a subject, comprising measuring expression of one or more of: hsa-miR-199a* and hsa-miR-324-3p.

15. The method of claim 14, useful as a diagnostic tool for determining disease progression, or staging, of COPD.

16. The method of claim 14, useful as a diagnostic tool for determining disease progression, or staging, of COPD, and distinguishing one or more of: stage 1 vs stage 2; stage 1 vs stage 4; and stage 2 vs 4 stage.

17. A method of determining chronic obstructive pulmonary disease (COPD) in a subject comprising:
i) obtaining a sample from the subject,
ii) analyzing the sample for the expression of one or more biomarkers, and
iii) correlating the expression of the one or more biomarkers with COPD in the subject,
wherein, the biomarkers are selected from the group consisting of the miRNAs: hsa-miR-199a* and hsa-miR-324-3p.

18. The method of claim 17, wherein the sample is selected from the group consisting of lung tissue, frozen biopsy tissue, paraffin-embedded biopsy tissue, sputum, bronchoalveolar lavage (BAL), and combinations thereof.

19. The method of claim 12, wherein the sample is analyzed by one or more methods selected from the group consisting of micro array techniques, PCR amplification, RNA hybridization, in situ hybridization, gel electrophoresis, and combinations thereof.

20. The method of claim 17, including correlating the expression of these biomarkers to the presence of stage 1 COPD.

21. The method of claim 17, including correlating the expression of these biomarkers to the presence of stage 2 COPD.

22. The method of claim 17, including correlating the expression of these biomarkers to the presence of stage 4 COPD.

23. A method of diagnosing COPD in a subject, comprising:
quantifying or detecting the amount of one or more miRNAs selected from FIG. 1: hsa-miR-455, hsa-miR-199a*, hsa-miR-324-5p, hsa-miR-324-3p, hsa-miR-133a, hsa-miR-193a, hsa-miR-015b, hsa-miR-374, hsa-miR-017-5p, hsa-miR-203, hsa-miR-374 and hsa-miR-429, detectable in a test sample, and
comparing the amount of the at least one miRNA in the test sample with the amount present in a normal control biological sample from a normal, COPD-free, subject, where an increase in the level of the at least one miRNA being found in the test sample is indicative that the disease is either progressing or has initiated.

24. A kit for diagnosing and staging chronic obstructive pulmonary disease (COPD) in a subject, the kit comprising:
i) a substrate for holding a biological sample isolated from a human subject suspected of having COPD,
ii) an agent that specifically binds at least one or more of the diabetic proteins; and,
iii) printed instructions for reacting the agent with the biological sample or a portion of the biological sample to detect the presence or amount of at least one marker in the biological sample.

25. The kit of claim 24, wherein the substrate can be hydrophobic, hydrophilic, charged, or polar.

26. The kit of claim 24, wherein the adsorbent is an antibody, single or double stranded oligonucleotide, amino acid, protein, peptide or fragments thereof.

27. The kit of claim 24, wherein one or more protein biomarkers is detected using immunoassays.

28. The kit of claim 24, wherein the immunoassay is an ELISA.
